(19) United States
(12) Patent Application Publication
(10) Pub. No.: US 2026/0198807 A1
(43) Pub. Date: Jul. 16, 2026

(54) GLUCOSE MONITORING

(71) Applicant: Whoop, Inc., Boston, MA (US)

(72) Inventors: Mostafa Ghannad-Rezaie, Malden, MA (US); Behnoosh Tavakoli, Needham, MA (US)

(21) Appl. No.: 19/451,302

(22) Filed: Jan. 16, 2026

Related U.S. Application Data
(60) Provisional application No. 63/746,065, filed on Jan. 16, 2025.

(51) Int. Cl. A61B 5/145; A61B 5/00; A61B 5/1455; A61B 5/1495

(52) U.S. Cl. CPC A61B 5/14532; A61B 5/14546; A61B 5/1455; A61B 5/1495; A61B 5/681; A61B 5/7271; A61B 2560/0223; A61B 2560/0462; A61B 2562/0233

(57) ABSTRACT

A light source is tuned to a range of wavelengths selected for relatively high absorption by glucose, while an optical sensor uses a complementary filter that selectively absorbs in the same spectrum. This optical channel, in combination with a separate, unfiltered reference optical channel, supports a calculation of glucose concentration in target tissue based on a ratiometric comparison of measured light intensities. In embodiments, quantum dots or other techniques can be used to tune the optical spectrum of a light source, while glucose or a similarly absorbing material can be embedded in an optical potting material or the like to create a similarly tuned filter for a corresponding optical sensor. The supporting hardware may usefully be deployed in a wearable physiological monitor for continuous monitoring of glucose (or other target molecules) in the tissue of a user.

### RELATED MATTERS

This application claims priority to U.S. Prov. App. No. 63/746,065 filed on Jan. 16, 2025, the entire content of which is hereby incorporated by reference.

This application is also related to International Patent App. No. PCT/US26/11535 filed on Jan. 16, 2026, which claims priority to U.S. Prov. App. No. 63/746,065 filed on Jan. 16, 2025, where the entire content of each of the foregoing is hereby incorporated by reference.

### BACKGROUND

Glucose monitoring is useful for management of diabetes, as well as for anticipating and managing a range of metabolic disorders such as impaired glucose tolerance, insulin resistance, metabolic syndrome, and so forth. While minimally invasive techniques using wearable continuous glucose monitors have emerged to replace or supplement invasive techniques such as finger prick blood samples, there remains a need for an accurate, non-invasive technique for continuous blood glucose monitoring.

### SUMMARY

A light source is tuned to a range of wavelengths selected for relatively high absorption by glucose, while an optical sensor uses a complementary filter that selectively absorbs in the same spectrum. This optical channel, in combination with a separate, unfiltered reference optical channel, supports a calculation of glucose concentration in target tissue based on a ratiometric comparison of measured light intensities. In embodiments, quantum dots or other techniques can be used to tune the optical spectrum of a light source, while glucose or a similarly absorbing material can be embedded in an optical potting material or the like to create a similarly tuned filter for a corresponding optical sensor. The supporting hardware may usefully be deployed in a wearable physiological monitor for continuous monitoring of glucose (or other target molecules) in the tissue of a user.

In one aspect, a system for glucose detection includes: a housing for positioning against a human tissue to be measured for a glucose concentration; a light source coupled to the housing and directed toward the human tissue when the housing is placed for use against the human tissue, wherein: the light source provides light at a target spectrum including: a first range of wavelengths corresponding to a glucose spectrum, and a second range of wavelengths corresponding to a reference spectrum, wherein the glucose spectrum includes wavelengths having a higher absorption by glucose than the reference spectrum, and wherein, in the glucose spectrum, an optical absorption in the human tissue varies in proportion to the glucose concentration in the human tissue; a first optical sensor coupled to the housing, the first optical sensor configured to acquire optical intensity data in the first range of wavelengths of the glucose spectrum, the first optical sensor positioned to receive the light transmitted in a first direction from the human tissue when the housing is placed for use against the human tissue; an optical filter for the first optical sensor, the optical filter positioned between the first optical sensor and the human tissue when the housing is placed for use on the human tissue, and the optical filter including a mixture of glucose and an optical potting material that is transparent in the first range of wavelengths; a second optical sensor coupled to the housing, the second optical sensor configured to acquire optical intensity data in the second range of wavelengths of the reference spectrum, the second optical sensor positioned to receive the light transmitted in a second direction from the human tissue when the housing is placed for use against the human tissue; a first optical barrier between the light source and the first optical sensor; a second optical barrier between the light source and the second optical sensor; and a processor. The processesor may be configured to: control illumination of the human tissue with the light source, acquire a number of measurements including an intensity of the glucose spectrum at the first optical sensor and an intensity of the reference spectrum at the second optical sensor, and calculate the glucose concentration in the human tissue based on a ratio of the number of measurements.

The light source may include one or more light emitting diodes and a coating for the light source including a plurality of quantum dots configured to receive a first optical emission from the one or more light emitting diodes and emit a second optical emission in the glucose spectrum. The light source may include one or more light emitting diodes and a coating for the light source including a plurality of quantum dots configured to receive a first optical emission from the one or more light emitting diodes and emit a second optical emission in the reference spectrum. The first optical sensor and the second optical sensor may include grayscale optical intensity sensors that acquire a single intensity measurement over a range of wavelengths including the first range of wavelengths and the second range of wavelengths. The processor may synchronize a first illumination in the glucose spectrum with a first intensity measurement with the first optical sensor, and a second illumination in the reference spectrum with a second intensity measurement with the second optical sensor. The optical potting material may include an optically clear silicone. The optical potting material may include a solvent for embedding glucose in the optical potting material. The second optical sensor may be offset at a greater distance from a contact surface of the human tissue than the first optical sensor. The reference spectrum may correspond to an absorption spectrum of a first interfering molecule that absorbs light in the glucose spectrum as a function of concentration and has a variable concentration in the human tissue. The target spectrum may include a third range of wavelengths corresponding to at least one of a second glucose spectrum and a second reference spectrum. The housing may include a wearable physiological monitor.

In another aspect, a system for glucose detection described herein includes a light source configured to emit light toward tissue of a subject, when the system is placed for use on a contact surface of the tissue of the subject, at a target spectrum including a first range of wavelengths corresponding to a glucose spectrum and a second range of wavelengths corresponding to a reference spectrum, wherein the glucose spectrum may include wavelengths having a higher absorption by glucose than the reference spectrum; a first optical sensor positioned to receive the light emitted from the light source and transmitted by the tissue of the subject, the first optical sensor configured to acquire optical intensity data in the glucose spectrum; a second optical sensor positioned to receive the light emitted from the light source and transmitted by the tissue of the subject, the second optical sensor configured to acquire optical intensity data in the reference spectrum; an optical filter positioned between the first optical sensor and the tissue of the subject when placed for use, the optical filter configured to attenuate light transmitted to the first optical sensor within the glucose spectrum; and a processor configured to calculate a glucose concentration in the tissue based on light intensity measurements from the first optical sensor and the second optical sensor.

The light source may include a plurality of quantum dots tuned to emit light within at least one of the first range of wavelengths and the second range of wavelengths. The optical filter may include glucose embedded in an optically clear potting material. The processor may calculate the glucose concentration based on a ratio of the light intensity measurements from the first optical sensor and the second optical sensor. The processor may calculate the glucose concentration using at least one of a machine learning model, a calibrated linear regression model, and a lookup table.

In another aspect, a system for measuring concentration described herein includes: a light source configured to emit light toward a target material at a target spectrum when placed for use on a contact surface of the target material, the target spectrum including a first range of wavelengths corresponding to a measurement spectrum and a second range of wavelengths corresponding to a reference spectrum, wherein the measurement spectrum may include wavelengths having a higher absorption by a target molecule than the reference spectrum; a first optical sensor configured to measure a first intensity of light transmitted by the target material in the measurement spectrum; a second optical sensor configured to measure a second intensity of light transmitted by the target material in the reference spectrum; an optical filter positioned between the first optical sensor and the target material when placed for use, the optical filter configured to attenuate the measurement spectrum more than the reference spectrum; and a processor configured to calculate a concentration of the target molecule in the target material based on the first intensity and the second intensity.

The target molecule may include water. The target molecule may include cholesterol. The system may further include a wearable physiological monitor that includes at least the light source, the first optical sensor, the second optical sensor, and the optical filter.

In another aspect, a method for measuring glucose concentration as described herein includes: emitting light at a target spectrum from a light source toward a target material, the target spectrum including a first range of wavelengths in a glucose spectrum containing a peak of an absorption spectrum for glucose, and the target spectrum including a second range of wavelengths in a reference spectrum away from the peak of the absorption spectrum for glucose, wherein: the first range of wavelengths does not overlap the second range of wavelengths, a glucose absorption within the first range of wavelengths in the glucose spectrum varies in response to changes in a concentration of glucose in the target material, and the first range of wavelengths in the glucose spectrum may include wavelengths having a higher absorption by glucose than the reference spectrum; receiving the light at a first optical sensor, the first optical sensor positioned to receive the light from the light source transmitted in a first direction by the target material; receiving the light at a second optical sensor, wherein: the second optical sensor is positioned to receive the light from the light source transmitted in a second direction by the target material, a first optical path from the light source to the first optical sensor is isolated by one or more optical barriers from a second optical path from the light source to the second optical sensor, and the first optical path to the first optical sensor may include a filter configured to attenuate the first range of wavelengths of the glucose spectrum; acquiring a plurality of measurements of optical intensity, the plurality of measurements including a first measurement intensity within the first range of wavelengths of the glucose spectrum at the first optical sensor and a second measurement intensity within the second range of wavelengths of the reference spectrum at the second optical sensor; and calculating a concentration of the glucose in the target material based on a ratio of the first measurement intensity and the second measurement intensity.

The light source may include one or more light emitting diodes with a coating including a plurality of quantum dots, the plurality of quantum dots configured to receive a first optical emission from the one or more light emitting diodes and emit a second optical emission including at least a portion of the target spectrum. Emitting light at the target spectrum may include exciting a plurality of quantum dots with one or more light emitting diodes, wherein the quantum dots are tuned to a portion of the target spectrum. The second range of wavelengths may span a peak absorption of an interfering molecule at a wavelength outside the first range of wavelengths of the glucose spectrum. The filter may be positioned along the first optical path between a contact surface of the target material and the first optical sensor. The filter may include a mixture of an optical potting material and glucose. The first optical path may be shorter than the second optical path. The first optical sensor and the second optical sensor may be positioned at different distances from a contact surface of the target material to balance a gain of an optical signal from the light source at the first optical sensor and the second optical sensor.

In another aspect, there is disclosed herein a computer program product comprising computer executable code embodied in a non-transitory computer readable medium that, when executing on one or more computing devices, causes the one or more computing devices to perform the steps of: causing a light source to emit light at a target spectrum toward a target material, the target spectrum including a first range of wavelengths in a target absorption spectrum corresponding to a peak of an absorption spectrum for a target molecule, and the target spectrum including a second range of wavelengths in a reference spectrum away from the peak of the absorption spectrum for the target molecule, wherein: an absorption within the first range of wavelengths in the target absorption spectrum varies in response to changes in a concentration of the target molecule in the target material, and the first range of wavelengths in the target absorption spectrum may include wavelengths having a higher absorption by the target molecule than the reference spectrum; causing a first optical sensor to capture a first measurement of optical intensity within the first range of wavelengths of the target absorption spectrum, the first optical sensor positioned to receive the light from the light source radiated in a first direction by the target material; causing a second optical sensor to capture a second measurement of optical intensity within the second range of wavelengths of the reference spectrum, wherein: the second optical sensor is positioned to receive the light from the light source radiated in a second direction by the target material, a first optical path from the light source to the first optical sensor is isolated by one or more optical barriers from a second optical path from the light source to the second optical sensor, the first optical path may include a filter configured to attenuate the target absorption spectrum associated with the target molecule, and the second optical path bypasses the filter; and calculating a concentration of the target molecule in the target material based on a ratio of the first measurement and the second measurement.

The target material may include human tissue. The target molecule may include glucose. The target molecule may include water. The target molecule may include cholesterol. The light source, the first optical sensor, and the second optical sensor may be in a wearable physiological monitoring device. The one or more computing devices may include a processor of the wearable physiological monitoring device. The one or more computing devices may include a remote processing resource configured to receive data from the wearable physiological monitoring device and calculate the concentration of the target molecule.

In another aspect, a method for measuring concentration, as disclosed herein, includes emitting light at a target spectrum from a light source toward a target material, the target spectrum including a first range of wavelengths within a measurement spectrum including a peak of an absorption spectrum for a target molecule and a second range of wavelengths within a reference spectrum away from the peak of the absorption spectrum; receiving the light at a first optical sensor and a second optical sensor, wherein: the first optical sensor is positioned to receive the light along a first optical path from the light source radiated in a first direction by the target material, the second optical sensor is positioned to receive the light along a second optical path from the light source radiated in a second direction by the target material, and the first optical sensor may include a filter configured to attenuate the first range of wavelengths corresponding to the peak of the absorption spectrum for the target molecule; acquiring a plurality of measurements of optical intensity with the first optical sensor and the second optical sensor including a first measurement from the first optical sensor within the measurement spectrum and a second measurement from the second optical sensor within the reference spectrum; and calculating a concentration of the target molecule in the target material based on a ratio of the first measurement and the second measurement.

The target material include human tissue. The light source may include one or more light emitting diodes coated with quantum dots that emit light within the target spectrum in response to illumination from the one or more light emitting diodes. The filter may include the target molecule dispersed in an optical potting material for the first optical sensor.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 illustrates a window for optical detection of a target molecule.

FIG. 7 illustrates an emission spectrum and filter for optical concentration measurement.

FIG. 8 illustrates a device for optical concentration measurement.

FIG. 9 is a flow chart of a method for determining a concentration of a target molecule in a target material.

### DESCRIPTION

The embodiments will now be described more fully hereinafter with reference to the accompanying figures in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user body temperature once every ten minutes or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example increasing the sampling rate when signal variability increases, or during periods of relatively higher motion, or based on user input.

At the same time, continuous monitoring is not intended to exclude ordinary data acquisition interruptions such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data such as optical intensity signals, or processed data therefrom such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component or module of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as, DRAM, SRAM, EDO RAM), and so forth. Although not depicted, any of the devices or components described herein may include a computer-readable medium or other memory for storing program instructions, data, and the like.

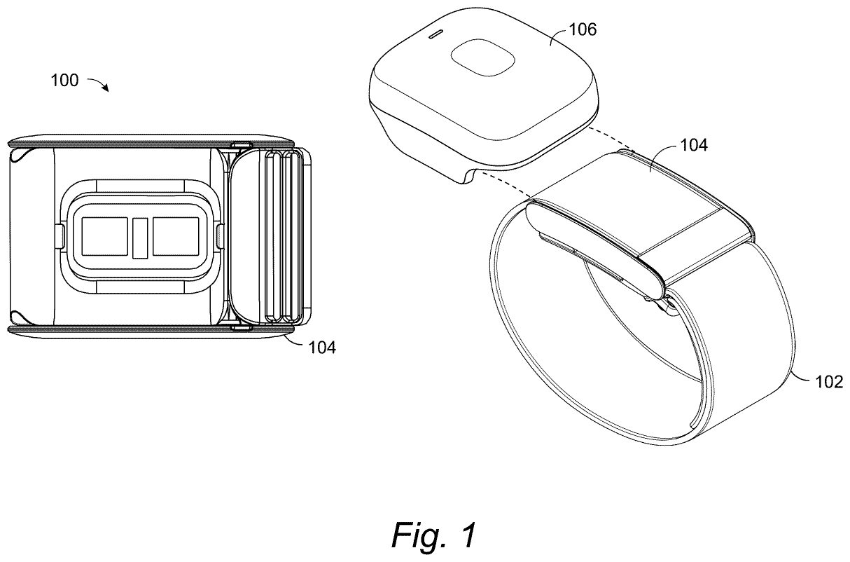

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104 and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure (via physical pressure measurements or other means), sound, electrocardiograms, and the like, as well environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting addition functions of the wearable monitor 104. For example, the system 100 may include communications systems to support, e.g., near field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

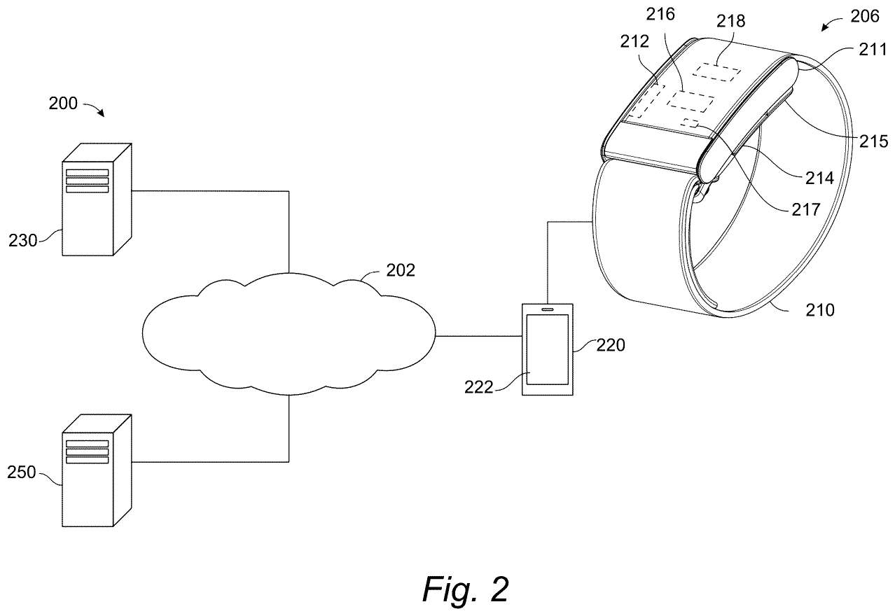

FIG. 2 illustrates a physiological monitoring system. More specifically, FIG. 2 illustrates a system 200 for physiological monitoring that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220 or otherwise supporting communicating with local resources. By way of non-limiting examples, short range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211 or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

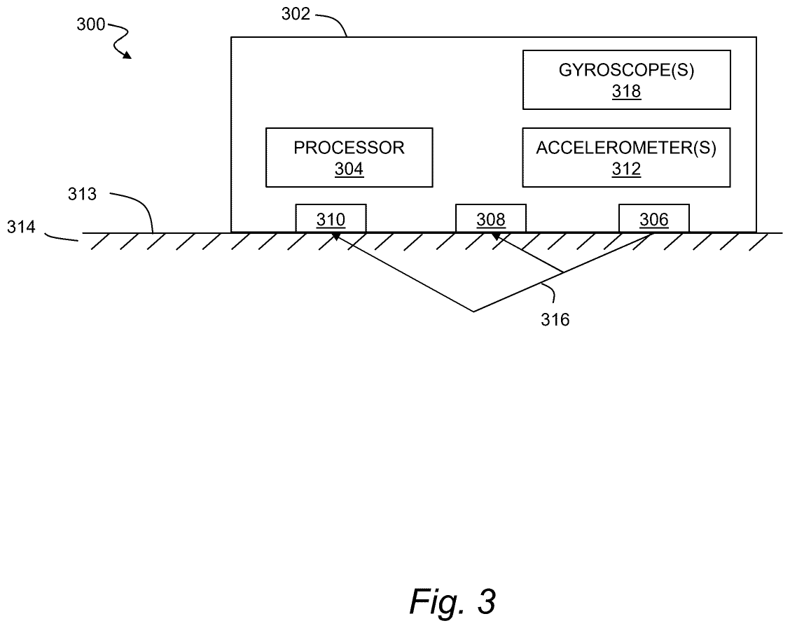

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like such as audio earbuds, earrings or similar, glasses and/or other eyewear, a ring, a headband, and so forth.

The processor 304 may be any microprocessor, microcontroller, application specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is transmitted back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

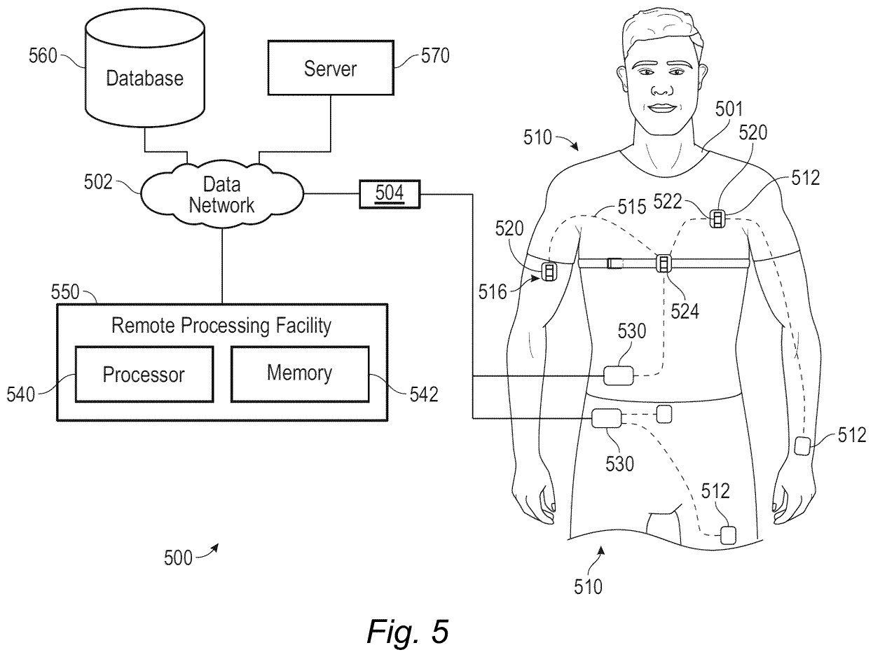

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

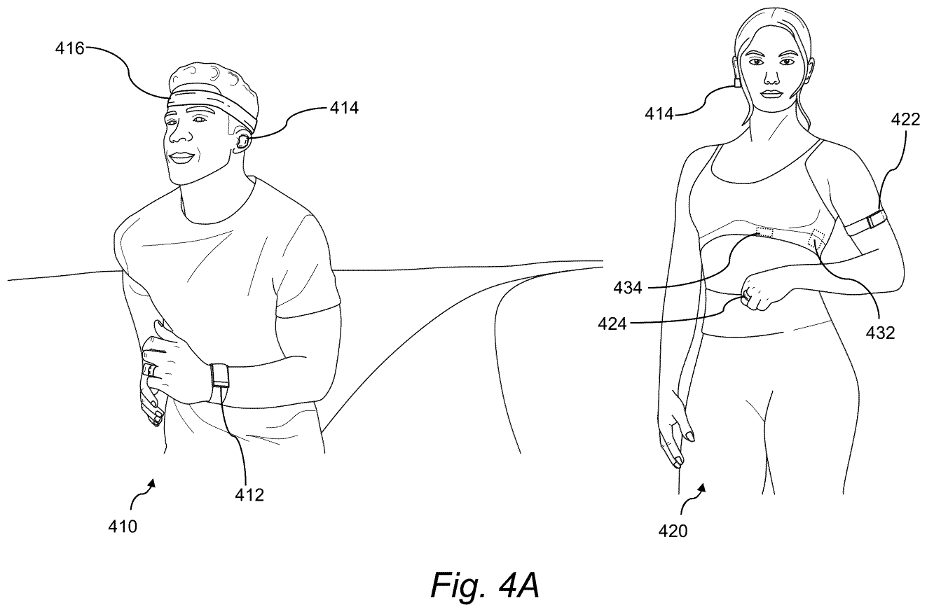

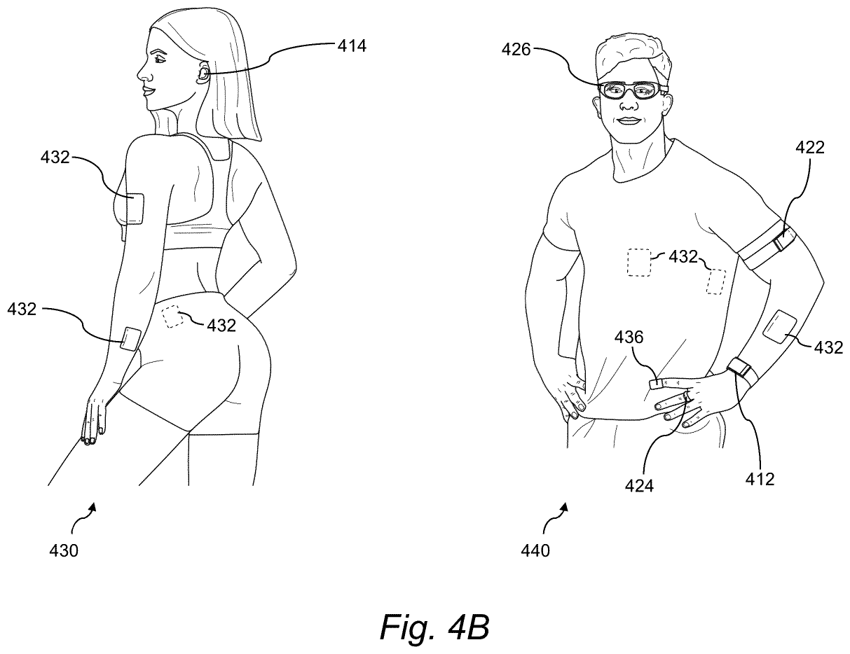

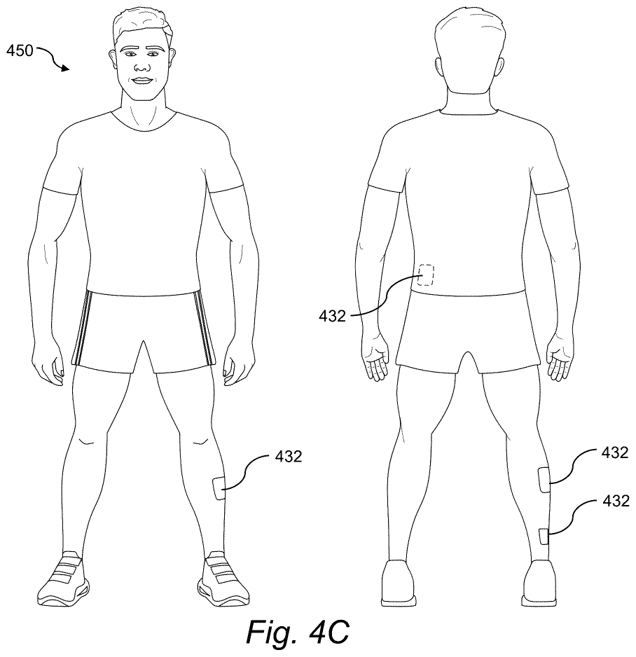

FIGS. 4A-4C illustrate physiological monitoring devices. The illustrated devices may include any of the hardware, software, and/or other components described herein for physiological sensing and/or other functions and may be embodied in various form factors for various use cases. These various form factors may be used individually or as multiple independent or cooperating physiological monitoring devices and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors such as a wrist-worn device 412 (such as any described herein), an ear-worn device 414 (including on-ear devices retained with a clamp, clip, or other mechanism, and/or in-ear devices such as earbuds or the like that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large scale motion such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors, and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to a pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smart phone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smart phone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as wi-fi or cellular signals, and converting them into usable electrical power), ambient light harvesting, and so forth. Other techniques may also or instead be used to provide external power, such as beam steering or resonant techniques for short range or medium range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based and may occupy a location that a user would prefer to reserve for other devices or jewelry, or that a user would prefer to leave unadorned for aesthetic or functional reasons. This location also places constraints on what measurements can be taken and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system to be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smart phone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for received light transmitted from the user's skin. The light detectors may include a photo-resistor, a phototransistor, a photodiode, and the like. A processor may process optical data from the light detector(s) to calculate a heart rate based on the measured, transmitted light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 524 may be any as described herein, for example including any of the features of the network interface 504 described above.

The controller 530 may be configured, e.g., by computer executable code or the like, to determine a location of the module 520. This may be based on contextual measurements such as accelerometer data from the module 520, which may be analyzed by a machine learning model or the like to infer a body position. In another aspect, this may be based on other signals from the module 520. For example, signals from sensors such as photodiodes, temperature sensors, resistors, capacitors, and the like may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500 such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510 and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510 and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes, and may, for example, be used to improve the design or use of modules 520 and garments 510, either for a population, for a user type, or for a particular user.

As stated above, the system 500 may further include a processor 540 and a memory 542. In general, the memory 542 may bear computer executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like as shown in the figure. Thus, in an aspect, one or more of the processor 540 and the memory 542 is included on at least one of the plurality of modules 520. In this manner, processing may be performed on a central module, or on each module 520 independently. In another aspect, one or more of the processor 540 and the memory 542 is remote relative to each of the plurality of modules 520. For example, processing may be performed on a connected peripheral device such as smart phone, laptop, local computer, or cloud resource.

The processor 540 may be configured to assess the quality of the data received from a physiological sensor 522 of the module 520, otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources, may advantageously be used in combination to improve physiological monitoring and achieve modes of monitoring not previously available.

In one aspect, any of the monitoring devices described herein may include a sensor system configured to optically monitor the concentration of a target molecule such as glucose by detecting selective optical absorption due to the presence of the target molecule in a measurement volume. Traditional glucose monitors rely on blood samples, or on invasive methods such as subcutaneous sensors, which can cause discomfort and require frequent replacement. At the same time, non-invasive optical spectroscopy methods such as laser-based spectroscopy, often considered the gold standard for molecular detection, has limitations for wearable devices due to its high cost and power requirements. The glucose monitoring techniques disclosed herein avoid these drawbacks by using quantum dots or the like to generate a custom, narrowband spectrum selected to match the absorption characteristics of glucose, in combination with an application-specific optical filter to support ratiometric measurements of glucose in target tissue.

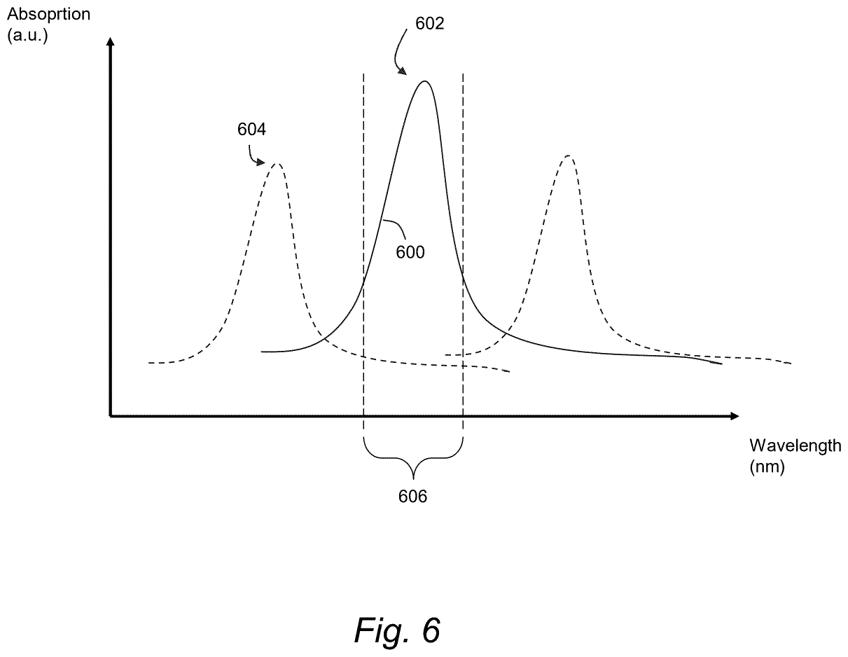

FIG. 6 illustrates a window for optical detection of a target molecule. As illustrated, the target molecule may have a first absorption profile 600 with a first absorption peak 602 (where light is maximally absorbed by the target molecule) that varies with concentration of the target molecule in a medium of interest. An interfering molecule (having an absorption that is detectable in a medium of interest) may have a second absorption peak 604 at a certain set of wavelengths above or below the first absorption peak 602. These aggregated optical properties provide a detection window 606 over which the presence of the target molecule in the medium can be detected based on the attenuation of an optical signal passing through the medium within the corresponding wavelength range. Furthermore, complementary measurements around peaks of adjacent interfering molecules can provide a useful baseline or reference for minimizing the effect on concentration measurements by the other interfering molecules in the target medium. This detection window 606 can more specifically be exploited as described herein to facilitate ratiometric detection of concentration of a target molecule such as glucose in human tissue.

It will be appreciated that the absorption profiles depicted in this figure are presented in arbitrary units. That is, the magnitude of absorption at any given wavelength is not necessarily to scale, or representative of any particular quantitative absorption metric or spectral profile. Rather, the absorption profiles depict the relative of absorption of different molecules at different wavelengths in order to illustrate the presence of a detection window 606 that can be used to measure concentration. It will also be appreciated that the actual absorption of any molecule at any wavelength will vary as a function of concentration of the corresponding molecule in a target material. This change in absorption may be approximated as a linear variation over relatively small changes and/or over certain ranges of concentration, or more generally as a proportional variation, e.g., that varies in direction with changes in concentration. In some cases, the change in absorption relative to concentration may be more complex and may vary according to other conditions such as temperature. Provided that the directional relationship persists over a useful range—e.g., a range where increases in concentration lead to increases in absorption within some practical optical measurement range for human tissue or other target material-then this effect may be exploited to measure concentration of a target molecule in a target material as described herein.

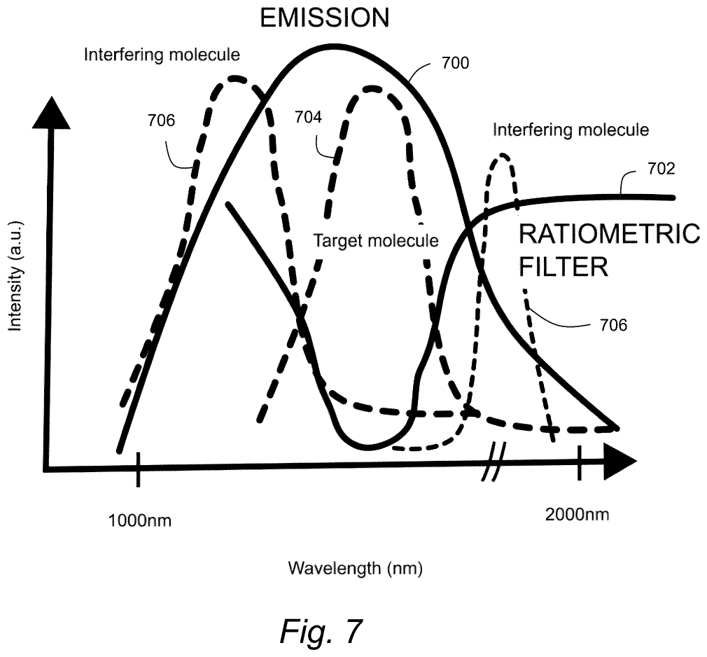

FIG. 7 illustrates an emission spectrum 700 and filter profile 702 for optical concentration measurement. In general, the emission spectrum 700 may be shaped, e.g., using quantum dots, filters, or the like, to provide range of wavelengths with a peak illumination intensity around the peak absorption spectrum 704 of the target molecule. The emission spectrum 700 may also be shaped to provide a range of wavelengths with a peak illumination intensity around a trough of the absorption spectrum 706 for one or more interfering molecules. This creates a physical sensing environment with an optical input signal focused on a spectrum that is relatively sensitive to the presence of the target molecule, and that is relatively insensitive to the presence of interfering molecules. In order to enhance specificity and minimize noise, a detection channel (in the detection window for the target molecule) may use optical filters tuned to the detection window generally, or at or around the absorption peak for the target molecule. Against this backdrop, intensity measurements through or off the surface of a target material may be captured over a measurement spectrum (with absorption sensitive to the target molecule) and a reference spectrum (with absorption that is relatively insensitive to the target molecule). The ratio of these signals can provide a robust measure of concentration of a target molecule that compensates for variations in tissue properties, optical coupling, and environmental factors.

In general, one optical sensor (the measurement sensor) may be used to measure the intensity of a signal in the measurement window that is transmitted from a target such as the skin of a subject and passing through a filter, while a another optical sensor may be positioned along a separate optical path that bypasses the filter and is used to measure the intensity of a signal outside the measurement window, which provides a reference intensity signal that is less influenced by the presence or absence of the target molecule in the target material that is being measured. A concentration of the target molecule may then be calculated based on a ratio of the filtered and unfiltered optical intensities, all as further described herein.

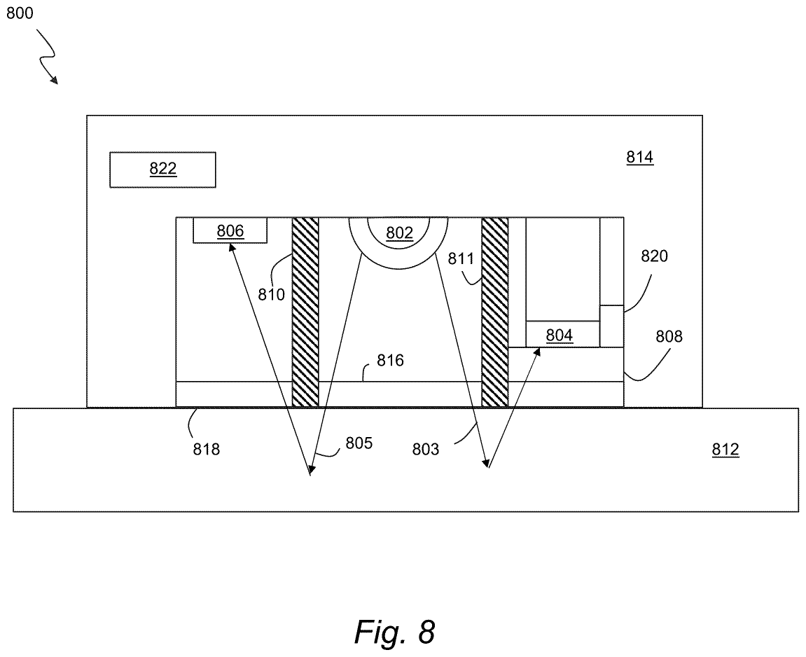

FIG. 8 illustrates a device for optical detection of the concentration of a target molecule. In general, the device 800 may include a light source 802, a first optical sensor 804, a second optical sensor 806, a filter 808 for the first optical sensor 804, optical barriers to separate from a target material 812 (such as human tissue or some other material from which a concentration measurement is desired) into separate optical paths, a housing 814, and an optical window 816. The light source 802 may be coupled to the housing 814 and directed toward a contact surface 818 of the target material 812 when the housing 814 is placed for use against the contact surface 818. The light source 802 may provide light over a spectrum for concentration measurements as described herein. In general, the spectrum for a ratiometric concentration measurement may include a first range of wavelengths (e.g., a target absorption spectrum or measurement spectrum) overlapping an absorption peak for the target molecule, along with a second range of wavelengths (e.g., a reference spectrum) away from the absorption peak.

The light source 802 may include one or more light emitting diodes or other source(s) of broadband or narrowband light. The light source 802 may provide light at a target spectrum for optical detection as described herein. For example, this may include a first range of wavelengths corresponding to an absorption spectrum of the target molecule, e.g., the measurement spectrum. The target spectrum may also include a second range of wavelengths (a reference spectrum) corresponding to an absorption peak of at least one interfering molecule and/or separated from the measurement spectrum for the target molecule. In general, an interfering molecule may be any other molecule in the target material that can absorb and/or scatter light and thus affect the intensity of measurements at a detector/sensor in proportion to the concentration of the interfering molecule in the target material. More generally, the target spectrum may be selected so that it is sensitive to the presence of the target molecule (with measurements in the measurement spectrum) and can minimize the effect on concentration measurements by one or more interfering molecules (with measurements in the reference spectrum). In one aspect, the target spectrum may include a range of wavelengths at or near a wavelength (or range of wavelengths) of peak absorption for the target molecule, and away from the absorption spectrum of one or more interfering molecules (e.g., so that the detection window is at or near a range of wavelengths of low or minimum absorption for the one or more interfering molecules).

In one aspect, a custom target emission spectrum can be generated from the light source 802 by integrating a quantum dot (QD) coating onto a light-emitting diode (LED), advantageously leveraging the unique photoluminescent properties of quantum dots to tailor the spectral output of the device. Quantum dots, which are semiconductor nanocrystals, exhibit size-dependent emission wavelengths due to quantum confinement effects, allowing for precise control over the output color by adjusting the QD size and composition. In an embodiment, a layer of quantum dots can be deposited onto the LED as a thin film or embedded within a polymer matrix or other carrier to achieve uniform dispersion. An LED of the light source 802 can emit a primary excitation light, typically in the blue or ultraviolet range, which may excite the quantum dots, causing them to emit light at specific wavelengths corresponding to their bandgap energy. By selecting a combination of quantum dot sizes and concentrations, a wide range of customized spectra can be achieved, such as narrowband emissions, broadband white light generation for general illumination, or tailored spectral distributions for imaging applications. Thus, quantum dots tuned to emit light within the target spectrum (e.g., within the first range of wavelengths and/or the second range of wavelengths) may be usefully integrated into light emitting diode coatings for use as described herein and may be used more specifically to create a light source with a first range of wavelengths around a peak absorption range for a target molecule and a second range of wavelengths away from the peak absorption range in order to provide a reference signal for ratiometric calculations. While quantum dots provide a useful mechanism for tailored emission spectra, it will be appreciated that more generally, any light source capable of providing illumination to a target at two wavelength ranges selected for sensitivity to a target molecule and a non-target molecule, or more generally, for sensitivity and insensitivity to the target molecule, may be used as light sources for systems and methods as described herein.

In one aspect, the target spectrum may be selected based on the characteristics of the absorption windows of a target molecule and any interfering molecules. For example, in a glucose measurement system, the first range of wavelengths (e.g., a glucose spectrum, or more generally, a target absorption spectrum) may be selected to overlap a glucose absorption peak and avoid the peak absorption window of one or more interfering molecules. The second range of wavelengths (e.g., a reference spectrum) may be selected to target a wavelength range with a lower absorption of glucose, e.g., so that intensity is less affected by variations in glucose concentration. More generally, the second range of wavelengths may be selected to provide a reference signal that is less sensitive to a varying concentration of the target molecule. This technique may also or instead be used with additional target wavelengths and/or reference wavelengths to provide additional optical channels that support measurement and refinement in the presence of varying concentrations of interfering molecules as well as other changing external conditions.

In one aspect, quantum dots may be used to create a light source for optical measurement of glucose concentration. Glucose has a prominent absorption window around 2100 nm. Using quantum dots with emission spectra centered at 2100 nm for inclusion (i.e., sensitivity to glucose) and 1900 nm for exclusion (i.e., insensitivity to glucose), glucose can be selectively detected in a medium such as human tissue. More generally, an inclusive channel (an optical channel in the measurement spectrum) may overlap the absorption band for the target molecule. At the same time, an exclusive channel (an optical channel in the reference spectrum) avoids this glucose band while providing a useful reference by providing an indicator of the imaging context that remains sensitive to background tissue absorption and scattering independent from changes in the glucose concentration. While the description herein focuses on the measurement of glucose concentration, these techniques may be adapted to other target molecules of interest whose concentration in a medium might usefully be measured. For example, water has absorption peaks around 1450 nm and 1940 nm, and an intervening minimum around 1700 nm, thus permitting optical detection and measurement of tissue hydration using a ratiometric calculation as described herein with an inclusive channel for optical measurements around 1940 nm and an exclusive channel for optical measurements around 1700 nm. Hydration measurement has potential applications in fitness tracking and medical diagnostics. In another aspect, quantum dots may be used to create a light source for ratiometric measurements of cholesterol concentration, which has an absorption peak for inclusion around 1700 nm, and a useful detection window between 1600-1800 nm.

In an embodiment, the device 800 may be configured to measure cholesterol concentration as the target molecule. In this implementation, the light source 802 may be tuned such that an inclusive channel overlaps a cholesterol absorption band in tissue, for example within a window between about 1600-1800 nm that encompasses a peak near approximately 1700 nm. An exclusive channel may be selected outside the cholesterol band to remain relatively insensitive to cholesterol. The filter 808 for the first optical sensor 804 may be formed by embedding cholesterol or a cholesterol-mimicking absorber in an optically clear potting material to create a filter that selectively attenuates wavelengths corresponding to the cholesterol absorption spectrum, thereby enhancing sensitivity of the ratiometric calculation to changes in cholesterol concentration. The optical sensors may acquire intensity measurements for the inclusive and exclusive spectral ranges along isolated optical paths, and a processor may calculate cholesterol concentration using a ratiometric comparison between the inclusive and exclusive measurements, optionally with calibration to map the comparison to concentration units and with compensations for temperature, coupling, motion, and so forth. This approach enables non-invasive, continuous monitoring of cholesterol or other lipids when suitable absorption windows and reference channels are selected. More generally any target molecule with an absorption that varies according to concentration in a medium of interest, and that has a characteristic spectral absorption curve with a significant peak and a significant trough in the medium, is amenable to optical concentration detection within that medium using the techniques described herein.

It will be appreciated that, while a system is described using two ranges of wavelengths—a measurement spectrum and a reference spectrum—the system may also or instead use additional wavelength ranges. In one aspect, this may include a second measurement spectrum, e.g., for measuring the concentration of a second molecule of interest. In another aspect, this may include an additional reference wavelength used to improve accuracy of measurements by providing a second reference measurement outside the measurement spectrum for the target molecule. Thus, in general, the device 800 may use a target spectrum that includes a third range of wavelengths, e.g., corresponding to a supplemental glucose absorption spectrum (for accuracy) or an additional target molecule spectrum (for separately measuring a second concentration).

The first optical sensor 804 may be coupled to the housing 814 and may include a photodetector or other device that converts incident light across a range of wavelengths into an electrical signal to facilitate detection and measurement of optical radiation. In one aspect, the first optical sensor 804 may be configured to acquire optical intensity data in a first range of wavelengths corresponding to the inclusive channel and the measurement spectrum. The first optical sensor 804 may be positioned to receive light transmitted in a first direction from the contact surface 818 when the housing 814 is placed for use against the contact surface 818. In this context, it will be understood that light transmitted from the contact surface 818 more generally includes light from the contact surface 818 transmitted toward the first optical sensor 804. While some portion of light from the light source 802 may be immediately reflected by the contact surface 818, e.g., due to differences in indices of refraction between different types of optical media, the light leaving the contact surface may also generally include light scattered toward the sensor from various depths within the target material 812 (e.g., human tissue). Thus, phrases such as “light from the contact surface,” or “light reflected from the contact surface,” as used herein, will be understood to refer more generally to radiant flux, or in a directional sense, radiance (toward a sensor of interest) of light transmitted from a surface of the target material 812 toward a sensor, unless a different meaning is explicitly provided or otherwise clear from the context. In general, light may be emitted by a light source toward a target material and then transmitted by a surface of the target material toward a sensor where the intensity can be measured.

The second optical sensor 806 may also be coupled to the housing 814 and may include a photodetector or other device that converts incident light across a range of wavelengths into an electrical signal to facilitate detection and measurement of optical radiation. The second optical sensor 806 may be configured to acquire optical intensity data in a second range of wavelengths corresponding to the exclusive channel and the reference spectrum. The second optical sensor 806 may be positioned to receive light in a second direction (e.g., not toward the first optical sensor 804) from the contact surface 818 when the housing 814 is placed for use against the contact surface 818 of the target material 812.

In some embodiments, the first optical sensor 804 and the second optical sensor 806 may include grayscale optical intensity sensors that acquire a single intensity measurement over a range of wavelengths. Grayscale sensors are advantageous for the general measurement of optical intensity because they are cheaper and do not use filters or other optical hardware that might tend to reduce the sensitivity of a sensor to optical energy. These sensors also do not require spectral processing, additional hardware, or other more complex control and processing that might otherwise tax the design and operation of small, wearable consumer devices and the like. In this case, the light source 802 may usefully be configured to operate sequentially in the first range of wavelengths and the second range of wavelengths, so that a grayscale intensity can be independently measured in the inclusive and exclusive channels without cross contamination. In another aspect, the light source 802 may include two independently controllable light sources that can be activated, e.g., in sequence, when acquiring signals for the inclusive and exclusive channels.

The filter 808 may be positioned between the first optical sensor 804 and the contact surface 818 when the housing 814 is placed for use on the contact surface 818 of a target material 812. In general, the filter 808 may be any filter for attenuating light in a target molecule detection window of wavelengths as described herein, e.g., a range of wavelengths where light is absorbed by a target molecule in proportion to the concentration of the target molecule in the target material 812. The filter 808 may be formed using any of a variety of optical components and techniques that selectively attenuate specific wavelengths of light corresponding to the characteristic absorption peaks of glucose molecules (or any other target molecule). In an embodiment, the filter 808 can be engineered using thin-film deposition techniques, incorporating dielectric layers or plasmonic nanostructures to achieve high spectral precision.

In one aspect, the filter 808 may include glucose embedded in an optical potting material for the first optical sensor 804, such as an optically clear silicone base or other material that is transparent or substantially transparent to the first range of wavelengths for the inclusive channel. More generally, the filter 808 may be created by embedding a target molecule directly into a medium that is interposed between the first optical sensor 804 and the target material 812 (or, during manufacture, between the first optical sensor 804 and a window or other contact surface for placement in contact with the target material 812). A filter 808 that embeds the target molecule in this manner can provide significant technical advantages. For example, the resulting filter is inherently tuned to the (potentially complex) absorption spectrum of the target molecule. Furthermore, the resulting filter will tend to respond to exogenous factors in the same manner as the target molecule that is being measured in the target material, thus improving accuracy over a range of conditions. For example, where the target molecule has an absorption that varies with temperature, the filter 808 can tend to match these variations in the target material 812 in a manner that reduces the temperature dependency of the measurement.

With respect to temperature, the filter 808 may also or instead include a temperature sensor 820 for measuring a temperature of the filter 808 and/or surrounding material. As noted above, for some molecules such as water, absorption characteristics can vary significantly as a function of temperature. In this context (e.g., a target molecule with temperature-dependent absorption), it may be useful to measure a difference in temperature between the optical potting material and the target material 812 to account for this variation when calculating a concentration of the target molecule or otherwise measure one or more temperatures associated with the calculation of concentration in order to improve accuracy.

A suitable or optimum optical potting material may be selected based on a number of criteria. For example, the optical potting material may be selected based on a transmission of pre-determined ranges of wavelengths, where an optically clear potting material (within the pre-determined ranges of wavelengths) can reduce or eliminate interference of the potting material with measurements of optical intensity. The optical potting material may also or instead be selected based on the long-term stability of the material for the expected use case. Thus, for example, a material that is expected to degrade or cloud over time under expected temperature or other environmental conditions would not be suitable for long-term, continuous sensing applications. However, where they are less expensive, such materials might be suitable for a short term or disposable sensor. In another aspect, the optical potting material may be selected for chemical and/or mechanical compatibility with the target molecule, particularly where the target molecule is incorporated directly into the potting material to form the filter 808. For example, glucose is hydrophilic (e.g., dissolving or wetting easily in water) while silicone is hydrophobic. Thus, where the optical potting material for a glucose filter includes an optically clear silicone, an additional solvent might usefully be added to a mixture of material used to form the filter in order to functionalize the glucose for emulsion, dissolution, or other embedding in a matrix of the silicone. Similar techniques may be used for other mechanically, chemically, or optically mismatched optical potting materials.

A variety of other suitable optical potting materials are known in the art and may be used as a potting material for creating the filter 808 as described herein. For example, the potting material may include an optically clear silicone such as polydimethylsiloxane (PDMS) or platinum-cure optical-grade silicone (e.g., Dow Sylgard 184, Momentive RTV615). The potting material may also or instead include an optical grade epoxy potting compound such as EPO-TEK 301 or EPO-TEK 302-3M, a polyurethane, an acrylic, a polycarbonate, or a fluoropolymer. The selection of a particular potting material may depend on the spectral bands of interest, long-term stability (yellowing, outgassing), adhesion to sensors and housings, viscosity and cure profile for embedding filters (e.g., target-molecule dopants), and compatibility with temperature and humidity cycles under expected use conditions.

In one aspect, the first optical sensor 804 may be offset from a plane of the light source 802 along a first optical path 803 from the light source 802 so that the first optical sensor 804 is physically closer to the surface of the target material 812 than the second optical sensor 806. That is, a first optical path 803 from the light source 802 to the first optical sensor 804 may be shorter than the second optical path 805 to the second optical sensor 806. This can address signal intensity/gain mismatches due to the structural variations between the optical chains for the two sensors. For example, a potting material with glucose or some other target molecule, when used as a filter, may tend to attenuate overall optical intensity of a signal transmitted from the surface of the target material 812 to a photodetector. Therefore, a physical offset may be used for either or both of the optical sensors in order to adjust a length of the optical path from the contact surface 818, and a corresponding relative gain of the optical signal received by the optical sensors. More generally, the first optical sensor 804 may be offset relative to the second optical sensor 806 at a predetermined distance from the contact surface 818 (e.g., closer to the contact surface 818 than the second optical sensor 806), the light source 802, or both. Other broadband filters and/or analog front ends may also or instead be used in order to match a gain of the signals for the pair of optical sensors.

The device 800 may include one or more optical barriers to separate the optical channels for the first optical sensor 804 (the filtered, depleted, or measurement sensor) and the second optical sensor 806 (the unfiltered, undepleted, or reference sensor). This can help to ensure independent measurement of the filtered and unfiltered channels for purposes of ratiometric calculations, particularly in cases where the sensors are channelized for concurrent acquisition of the measurement and reference signals. This separation can also decrease the loss of light and optical energy under general illumination conditions. In an embodiment, a first optical barrier 810 may be positioned between the light source 802 and the first optical sensor 804, and a second optical barrier 811 may be positioned between the light source 802 and the second optical sensor 806. This provide optical separation of the first optical path 803 from the second optical path 805 to permit improved separation of measurements for each channel.

A housing 814 may incorporate components of the device 800, with the components coupled to the housing 814 in any suitable physical arrangement consistent with the systems and methods described herein. In general, the housing 814 may provide sealing/containment for use of the glucose monitor (or other measurement device) in different environments, as well as optical isolation to mitigate interference by ambient light with optical measurements. The housing 814 may also or instead incorporate any of the components and features of a physiological monitor as described herein and may include hardware suitable for retaining the housing 814 in a position on a user for continuous glucose monitoring. In use, the housing 814 may be positioned against a contact surface 818 of a target material 812 such as human tissue for an optical measurement of glucose concentration of a user, or more generally, may be positioned against a target material 812 for measurement of the concentration of a target molecule in the target material 812, all as more generally described herein.

The optical window 816 may be configured to allow light to be emitted from light source 802 into the target material 812 and to be transmitted by the target material 812 to the first optical sensor 804 and the second optical sensor 806, e.g., along a first optical path 803 and a second optical path 805. In general, the optical window 816 may be formed of any material(s) or the like that can seal an interior of the housing 814 while permitting optical sensing therethrough when the optical window 816 is placed in contact with a target surface such as the contact surface 818 of the target material 812. For example, the optical window 816 may be formed of an optically clear material such as polycarbonate or the like, and may include filters, filtering materials, surface features, lenses or the like to augment the channelized, differential filtering between the first optical sensor 804 and the second optical sensor 806 as described herein.

A processor 822, such as any of the processors or other processing circuitry described herein, may be used to control illumination of the contact surface 818 from the light source 802 and to acquire data from the optical sensors for calculation of a concentration of the target molecule. This may include acquiring a number of measurements to support ratiometric calculation of the concentration, such as an intensity measurement in the first range of wavelengths (the inclusive channel for the target absorption spectrum) and the second range of wavelengths (the exclusive channel away from the target molecule peak), which may be captured at the first optical sensor 804 and the second optical sensor 806 respectively.

In one aspect, the intensity data may be transmitted to another processing resource for a calculation of concentration of the target molecule. In another aspect, the processor 822 may be configured to calculate a glucose concentration for tissue beneath the contact surface 818 based on a ratio of the number of measurements. In general, the optical intensity measurements used to support a ratiometric inference of concentration will include two different measurements: a first measurement in the absorption window for the target molecule, and a second measurement outside the absorption window, each of which are acquired at one of the sensors. In general, this may be accomplished in a number of ways. For example, the light source 802 may be controlled to illuminate first with one range of wavelengths (e.g., the inclusive channel, or within the range of wavelengths attenuated by the target molecule) and then with another range of wavelengths (e.g., the exclusive channel, or a range of wavelengths less attenuated by the target molecule). By time-separating these two channels, suitable intensity measurements can be captured with synchronized time-separated measurements. This approach may advantageously simplify the detector hardware by permitting use of grayscale photodetectors or other non-spectral sensors of optical energy. However, in another aspect, the optical sensors may be configured for dual band sensing, e.g., with each of the optical sensors matched to a corresponding range of wavelengths. In this case, the first optical sensor 804 may include a first input filter that is optically band limited to the measurement spectrum and the second optical sensor 806 may include second input filter that is band limited to the reference spectrum. For simplicity of illustration, these filters are not included in FIG. 8, however, they may be deployed as thin films on the sensors, on the optical window 816, or using any other suitable physical filtering techniques, and or by postprocessing the output from multi-spectral optical sensors. In any case, the resulting inclusion and exclusion measurements may be used as further described herein to support a ratiometric calculation of the concentration of a target molecule.

In general, the first optical sensor 804 will detect an intensity of light that is proportional to the intensity of the light source 802, but that varies with the concentration of the target molecule within the target material 812. At the same, the second optical sensor 806 will detect an intensity of light that might also vary with concentration of the target molecule, but in a manner that is generally less sensitive to the concentration of the target molecule in the target material 812. Due to this relative decoupling of the signal at the second optical sensor 806 from the presence of the target molecule, the signal from this second optical sensor 806 may serve as a reference for evaluating changes in intensity of the signal received at the first optical sensor 804, which will have a component that is more responsive to variations in the concentration of the target molecule in the target material 812. Thus, the signal received at both the first optical sensor 804 and the second optical sensor 806 may increase or decrease as a function of the target molecule, however, the rate of attenuation (relative to concentration of the target molecule) at both sensors will be different, enabling an inference about concentration based on a ratio of two corresponding intensity measurements. This configuration advantageously supports a ratiometric calculation that compensates for variations in tissue properties, optical coupling, and environmental factors, thereby isolating the target-specific signal and enabling a calibration to physical metrics (such as mg/dL for blood glucose). Additionally, the use of a target-enriched optical filter further enhances target-specific absorption in the measurement channel, increasing sensitivity and precision.

In general, any technique for comparing intensity over two wavelength ranges from the two photodiodes, including ratiometric calculations, linear regression models, machine learning models, lookup tables, empirical models, and so forth, may be used to optically evaluate a concentration of a target molecule such as glucose. The result may be expressed as a relative change, or, with suitable calibration data, as absolute values of concentration, which may be expressed in any suitable units of concentration. For example, human blood glucose concentrations are commonly expressed in mg/dL, with a typical fasting concentration of about 70-100 mg/dL and a postprandial (post-meal) concentration of up to 140 mg/dL. By way of further parameterization, severe hypoglycemia is often considered <54 mg/dL, and random diabetic tests can exceed 200 mg/dL. Tissue (interstitial fluid) values closely track blood with a small lag and minor offsets. Measurements may be calibrated to these quantitative measures of glucose concentration for an individual or, where calibration parameters are consistent among subjects, for general users.

The calculation of concentration may be adjusted or refined based on environmental or contextual factors. For example, the calculation may be adjusted to account for absorption of the target spectrum by the optical potting material, a difference in homogeneity of the target material, or a difference of the temperature of the target molecule in the optical potting material, the temperature of the target molecule in the target surface, the presence of interfering molecules, and so forth. Other factors such as blood flow of a user may also affect an optical measurement of the concentration of a target molecule such as glucose and may be measured and used to adjust calculations of concentration, which are described in greater detail below.

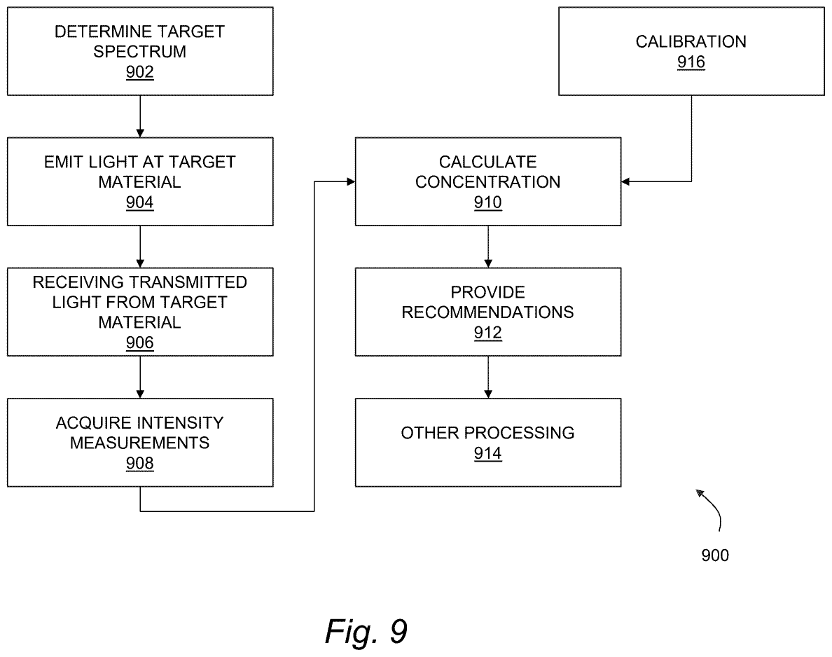

FIG. 9 is a flow chart illustrating a method of determining a concentration of a target molecule in a target material. The method 900 may be deployed, e.g., in any of the physiological monitoring devices described herein. In this context, determining a concentration may include continuously monitoring the concentration, e.g., in the same continuous manner as heart rate and other physiological metrics.

As shown in step 902, the method 900 may include determining a target spectrum. Determining a target spectrum may include selecting one or more ranges of wavelengths for preferential inclusion and exclusion of a target molecule and/or an interfering molecule, all as described herein. In one aspect, determining a target spectrum may include selecting a first range of wavelengths for the target spectrum corresponding to an absorption spectrum of the target molecule (e.g., a range containing a peak absorption by the target molecule) and at least one second range of wave lengths corresponding to an interfering molecule (e.g., outside the first range of wavelengths). Additional ranges of wavelengths may be selected corresponding to an absorption spectrum of additional target molecules, and/or to provide additional reference signals for improved accuracy. In general, the target spectrum may be selected to support detection of the presence of the target molecule and minimize the effect on concentration measurements by one or more interfering molecules. The determination of the target spectrum may be performed using stored spectral data, empirical measurements, calibration data, models, or any combination thereof, and may be specific to a particular target molecule, target material, Interfering molecules, measurement conditions, and so forth.

In another aspect, determining a target spectrum may include selecting at least one range of wavelengths for the target spectrum based on a comparison of an absorption spectrum of the target molecule of the target material and the absorption spectrum of at least one interfering molecule. For example, a first range of wavelengths may be selected where the absorption spectrum corresponding to the target molecule exhibits a high absorption relative to the at least one interfering molecule and/or at a peak of the absorption spectrum for the target molecule. A second range of wavelengths may be selected where the target molecule exhibits relatively lower absorption compared to an interfering molecule and/or where an interfering molecule has an absorption peak and/or over a range of wavelengths separated from the first range of wavelengths.

By windowing the ranges of wavelengths in this manner, a system can support differential or ratiometric estimations of the target molecule. Thus, the determined spectrum may be used to create a suitable light source, filter, and optical sensors for acquiring a plurality of intensity measurements to determine a concentration of the target molecule in a target material. This hardware, along with a processor or other processing circuitry for control and calculation, may be housed, e.g., in a wearable physiological monitoring device such as any of the devices described herein, or any other device for measuring concentration, and/or that might benefit from non-invasive concentration measurements within a target material. Once the spectrum has been determined and corresponding hardware prepared or provided, concentration measurements may be obtained as further described herein.

As shown in step 904, the method 900 may include emitting light at a target spectrum from a light source toward a target material such as human tissue.

For glucose monitoring, the target spectrum may include a first range of wavelengths in a glucose spectrum corresponding to a peak of the absorption spectrum for glucose and a second range of wavelengths in a reference spectrum away from the peak of the absorption spectrum for glucose. In embodiments, the second range of wavelengths may span a peak absorption of an interfering molecule at a wavelength outside the first range of wavelengths, thereby providing a reference channel that is relatively insensitive to glucose (compared to the glucose spectrum) while remaining responsive to background absorption and scattering. The light source may, for example, include one or more light emitting diodes with a coating including a plurality of quantum dots tuned to the target spectrum. These quantum dots may receive a first optical emission from the diodes and emit a second optical emission including at least a portion of the target spectrum.

In another aspect, the light source may sequentially excite different pluralities of quantum dots tuned to the first and second ranges of wavelengths to support inclusive and exclusive optical channels for time-multiplexed acquisition. In general, absorption of the light by glucose within the first range of wavelengths in the glucose absorption spectrum may vary in response to changes in a concentration of glucose in the target material. Thus, the absorption of the bulk material will vary in response to changes in concentration of glucose. While this property can provide a general directional indicator of changes in glucose concentration, additional illumination and measurements may be used as described herein to derive a quantitative estimate of glucose concentration. In another aspect, the first range of wavelengths in the glucose spectrum includes wavelengths having a higher absorption by glucose than the reference spectrum. This permits a differential reference channel to be provided in a spectrum outside the first range of wavelengths.

The techniques described herein may be used to measure glucose concentrations in human tissue. Thus, the target material may include human tissue. The techniques described herein may also or instead be used to measure concentrations of other target molecules. Thus, the method 900 may more generally include emitting light in a target spectrum including a first range of wavelengths in a target absorption spectrum corresponding to a peak of the absorption spectrum for a target molecule and a second range of wavelengths in a reference absorption spectrum away from the peak of the absorption spectrum for the target molecule. The target molecule may include glucose as described above, or other molecules of interest. For example, the target molecule may include water (e.g., for measuring tissue hydration) or cholesterol.

As shown in step 906, the method 900 may include receiving the light transmitted by the target material. For example, this may include receiving light at a first optical sensor positioned to receive the light from the light source transmitted in a first direction by the target material. The first optical sensor may be configured to acquire optical intensity within the first range of wavelengths. In one aspect, this may include providing illumination in the first range of wavelengths while acquiring intensity data with the first optical sensor, thus creating a time slot or time segment for the measurement spectrum, followed by illumination in the second range of wavelengths while acquiring intensity data with the second optical sensor. In these embodiments, the first optical sensor and/or the second optical sensor may be grayscale optical intensity sensors that acquires a single intensity measurement over the active wavelength range during each emission interval. In another aspect, each sensor may include a band-filtered input to selectively acquire optical intensity data within the relevant wavelength range. While potentially increasing the cost and complexity of system hardware, this may also advantageously enable concurrent acquisition of inclusive and exclusive channel signals.

Receiving the light mays also receiving light at a second optical sensor positioned to receive the light from the light source transmitted in a second direction by the target material. A first optical path from the light source to the first optical sensor may be isolated by one or more optical barriers from a second optical path from the light source to the second optical sensor to prevent crosstalk and preserve independent channel measurements. The first optical path may include a filter configured to attenuate the first range of wavelengths of the glucose absorption spectrum, and in embodiments, the filter may be positioned along the first optical path between a contact surface of the target material and the first optical sensor. In embodiments, the filter includes a mixture of an optical potting material and glucose to create an application-specific absorber that selectively depletes photons in the glucose band while transmitting the reference band. To balance signal levels between channels, first and second optical sensors may be positioned to balance a gain of the optical signal from the light source at each sensor. Thus the first optical path may be shorter than the second optical path in order to increase the gain of the signal on the first sensor so that the filtered channel maintains a greater dynamic range despite attenuation by the filter.

As shown in step 908, the method 900 may include acquiring a plurality of measurements of optical intensity with the first and second optical sensors. The plurality of measurements may include a first measurement intensity within the first range of wavelengths of the glucose spectrum at the first optical sensor and a second measurement intensity within the second range of wavelengths of the reference spectrum at the second optical sensor. More generally, acquiring the plurality of measurements may include acquiring a plurality of measurements of optical intensity with the first optical sensor and the second optical sensor to support ratiometric calculations of concentration. In various configurations, acquisition may be synchronized with emission so that inclusive and exclusive measurements are obtained in successive intervals, or band-selective detectors may be operated concurrently.

As shown in step 910, the method 900 may include calculating a concentration of glucose in the target material based on a ratio of the plurality of measurements.

In one aspect, an effective optical model for concentration estimation in tissue assumes that the detected signals in each spectral channel can be represented by a lumped-path description of light transport. In this model, photons contributing to a measurement traverse an effective optical path length L_{eff} through the target material, and attenuate due to background absorbers and scattering, as well as due to the presence of a target molecule of interest. As described herein, a light source may form two channels: an inclusive channel whose spectrum overlaps the absorption band of the target molecule and an exclusive channel whose spectrum avoids that band and is therefore insensitive to a varying concentration of the target molecule while remaining responsive to background attenuation. Ratiometric analysis compares these channels to isolate the effect of target concentration and suppress other context-dependent variations, e.g., due to motion, tissue deformation, temperature, and so forth.

As described herein, a light source may emit two separated spectral ranges using a plurality of quantum dots tuned to an inclusive band that overlaps a peak in the target absorption spectrum and an exclusive band in a reference spectrum separated from that peak. The emitted illumination enters a target material such a tissue through a window, and radiance from the target material is measured along two isolated optical paths by first and second optical sensors. The inclusive path may incorporate a detection-side filter enriched with the target molecule to selectively deplete photons in the target band, thereby increasing spectral selectivity and steepening the sensitivity of the inclusive channel to concentration changes. Optical barriers may be used to separate the paths and prevent cross-channel contamination. Grayscale detectors may be used with time-multiplexed emission to measure band-specific intensities, or band-selective detectors may be employed concurrently.

Under quasi-static conditions during a measurement, the coupling, geometry, and background optical properties can be assumed constant. The inclusive and exclusive signals can then be modeled by an effective Beer-Lambert relation where, for channel i∈{inc,exc}, the detected signal Sᵢ can be expressed as:

$${S}_{i} = {S}_{i , 0} exp ( - {μ}_{bg , i} {L}_{eff} - {ϵ}_{t , i} {C}_{t} {L}_{eff} ) ,$$

where
   S_{i,0} is the effective source intensity and detector gain for channel i,
   μ_{bg,i} is the effective background attenuation coefficient for non-target absorbers and scattering at the wavelengths of channel i,
   ϵ_{t,i} is the effective extinction coefficient of the target molecule for channel i, and
   Cₜ is the target concentration to be estimated.

The exclusive channel is selected to be target-insensitive, so the effective extinction coefficient approaches zero, or:

$${ϵ}_{t , inc} \approx 0$$

yielding:

$${S}_{exc} = {S}_{exc , 0} exp ( - {μ}_{bg , exc} {L}_{eff} ) ,$$

$${S}_{inc} = {S}_{inc , 0} exp ( - {μ}_{bg , inc} {L}_{eff} - {ϵ}_{t , inc} {C}_{t} {L}_{eff} )$$

Taking the natural logarithm of both signals and subtracting forms a log-ratio where:

$$ln ( \frac{{S}_{exc}}{{S}_{inc}} ) = ( ln {S}_{exc , 0} \text{?} - {μ}_{bg , e} \text{?} + {ϵ}_{t , inc} \text{?}$$

$$\text{?} \text{indicates text missing or illegible when filed}$$

Solving explicitly for the target concentration and grouping constants yields a relationship of the general form:

$${C}_{t} = \frac{1}{{β}_{1}} [ ln ( \frac{{S}_{exc}}{{S}_{inc}} ) - ln ( \frac{{S}_{exc , 0}}{{S}_{inc , 0}} ) ] - {β}_{0}$$

Further, under constant conditions without noise, the effective source intensities may also be treated as constant, further simplifying this relationship to:

$${C}_{t} = \frac{1}{{β}_{1}} [ ln ( \frac{{S}_{exc}}{{S}_{inc}} ) - {β}_{0} ]$$

In practice, the constant terms can be resolved and absorbed into calibration parameters where β₀ and β₁ are obtained from reference measurements. For improved flexibility over wider ranges or when mild nonlinearity is observed, a polynomial mapping can be used:

$${C}_{t} = {a}_{0} + {a}_{1} ln ( \frac{{S}_{exc}}{{S}_{inc}} ) + {{a}_{2} [ ln ( \frac{{S}_{exc}}{{S}_{inc}} ) ]}^{2} +$$

with coefficients a₀, a₁, a₂, a₃, . . . determined empirically. A variety of other techniques may be used to deploy this relationship for resolving concentration based on optical measurements. For example, a processor may use a nonparametric model, lookup-table, linear regression model, and/or machine-learned models to calculate absolute concentrations based on this relationship and a variety of calibration data or reference measurements.

Implementation details may also include emission control to time-multiplex the inclusive and exclusive spectra, synchronization of acquisition at the detectors, and preprocessing steps such as dark-current subtraction, ambient-light rejection, normalization for source intensity variation, and balancing of optical path gains. The processor may compute the comparison between channels, apply the calibrated mapping to obtain concentration, and optionally compensate for temperature, detector responsivity drift, motion artifacts, coupling variability, and the like. The computation may be performed locally on a wearable device or remotely on a processing resource receiving the measured intensities. Continuous operation of emission filtering and ratiometric analysis as described herein may advantageously provide time-resolved estimates of target concentration with robustness to background absorbers (e.g., interfering molecules) and changes in optical coupling.

Other variations of hardware, software, and processing algorithms may be used to measure the concentration of a target molecule as contemplated herein. For example, a concentration monitor based on emission filtering and ratiometric analysis can be implemented in a variety of configurations that preserve the core relationship between the inclusive and exclusive channels while adapting the optics, detection, and computation for different constraints. In one embodiment, a single detector is used with time-multiplexed emission, where the light source alternates between the inclusive and exclusive spectra and the detector captures synchronized, band-filtered measurements for each interval. This approach reduces component count and simplifies gain matching, while maintaining the logarithmic ratio of exclusive and inclusive channels needed for concentration estimation. In another embodiment, two detectors are used concurrently with band-selective detection, where each detector measures only one channel while a common source emits a composite spectrum. Optical barriers and physical separation preserve path isolation, and electronic gain and path-length adjustments may be used to bring both channels into comparable dynamic ranges.

In another aspect, source-side spectral selectivity may be achieved with a variety of techniques including quantum-dot coatings, narrowband LEDs, superluminescent diodes, or compact tunable sources such as MEMS-tunable filters and micro-Fabry-Perot cavities placed in front of broadband emitters. Detection-side selectivity may use thin-film dielectric bandpass filters, plasmonic metasurface filters, or an application-specific absorber in an optically clear potting material that embeds or mimics the target molecule. In further embodiments, a mosaic detector or multispectral camera may use pixel-level filter arrays to form spatially multiplexed inclusive and exclusive channels over an imaging field, with the ratio computed per pixel and aggregated for a region of interest to estimate concentration.

Geometrically, the system may operate in reflective mode with emitters and detectors co-located on the same side of the tissue, in transmissive mode across a thin anatomical site such as a fingertip or earlobe, or with fiber-coupled probes that deliver and collect light at controlled separations to tune effective path length. Path gain balancing may be implemented mechanically by sensor standoff and window geometry, optically by neutral-density or gain-equalizing filters, or electronically by detector transimpedance gain and source current control, with closed-loop algorithms maintaining target signal amplitudes in a calibrated operating range.

In general, computation can be used to map the comparison of intensity ratios, r, to concentration using calibration. A linear parametric form C=(r−β0)/β1 may be used over ranges where the effective Beer-Lambert description is sufficiently linear. For wider ranges or mild nonlinearity, a low-order polynomial (e.g., as described above) may be used to improve fit while remaining interpretable. In other implementations, nonparametric lookup tables or machine-learned regressors (e.g., Gaussian processes, gradient boosting, or small neural networks) may be used to predict concentration together with auxiliary variables such as temperature, coupling metrics, and motion features. Multi-channel extensions may incorporate additional spectral bands to jointly estimate a vector of concentrations for multiple target molecules with a coupled model, or to improve glucose accuracy by explicitly modeling multiple interfering absorbers; in these cases, the comparison may become a feature vector formed from several ratios or band intensities, where the estimator solves a small inverse problem or applies a multivariate regression.

System integration may be local or distributed. In one aspect, a wearable device locally performs emission control, synchronized acquisition, preprocessing, ratio formation, and concentration estimation, storing results to local memory and presenting values through a user interface with alerts on threshold crossings. Alternatively, raw or preprocessed intensities may be transmitted to a remote processing resource that executes the concentration calculation, manages longitudinal analytics, and returns estimates for display and recommendations. In continuous monitoring, the device may schedule periodic calibration checks, maintain closed-loop source control to hold signal levels within desired bounds, and automatically adapt sampling rates based on motion, signal quality, user input, and so forth.

As shown in step 912, the method 900 may include providing a recommendation to a user. A recommendation may be provided to the user based on the calculation of the concentration of the target molecule. For example, recommendations may be provided to user based on how the calculation compares to expected or preferred values of the target molecule. In general, recommendations may be based on global guidelines, recent measurements for a user, or a history of measurements for the user, or some combination of these. Depending on the molecule measured and quantitative value of the measurement, recommendations may include long term recommendations (e.g., behavioral modifications, diet changes, exercise plans) and/or short term recommendations for immediate action (e.g., rest, don't eat sugary foods or simple carbohydrates, or hydrate immediately). For example, a concentration of water in a user reflects the hydration of a user. Therefore, a recommendation may be provided to the user to increase their water intake if the concentration of water suggests they are dehydrated. In another example a concentration of glucose over a threshold may trigger a recommendation for a short term remediation (e.g., go for a walk) and/or a longer term remediation (starting meals with fiber). The recommendation may also be based on additional factors known about the user such as a health history of the user or a diet of the user. For example, different recommendations may be given to a user for a particular glucose concentration if they are diabetic. In general, recommendations may be provided to the user based on how the calculation of the target molecule corresponds to target values for the concentration and may further account for factors such as user-specific conditions or history. In another aspect, thresholds may trigger notifications to a coach, clinician, or caregiver, e.g., to improve the quality of care or provide professional advice.

As shown in step 914, the method 900 may include other processing. For example, this may include storing a current concentration measurement in a history for the user or exporting the concentration data to an external data store, data service, or the like for analysis or future use. Processing may also or instead include displaying the calculated value to a user, and/or displaying longitudinal data for the user such as a historical graph of measurements to provide context for the current measurement. More generally, any useful post-measurement steps may be performed after a measurement is obtained.

As shown in step 916, the method 900 may include calibrating a system that performs concentration calculations. In one aspect, this may include acquiring a collection of measurements to calibrate a model such as any of those described herein for calculation of absolute concentration values. In another aspect, this may include interim calibrations to account for movement of sensors, tissue deformation (e.g., due to changes in a strap tightness or placement of the system), and so forth. In one aspect, a second reference channel may be used to refine a mathematical model, e.g., by providing sufficient constraints to directly calculate a calibration term such as the effective length of an optical path (L_{eff} above). In general, this approach permits reuse of constants from a general calibration, with local adjustments based on available inferences about the measurement context, and can support accurate, absolute calculations under varying use conditions. More generally, any global or local calibration techniques known in the art, along with additional sensing hardware and supporting control and calculations as needed, may be used to improve accuracy of the systems and methods described herein.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A system for glucose detection, the system comprising:
a housing for positioning against a human tissue to be measured for a glucose concentration;
a light source coupled to the housing and directed toward the human tissue when the housing is placed for use against the human tissue, wherein:
the light source provides light at a target spectrum including:
a first range of wavelengths corresponding to a glucose spectrum, and
a second range of wavelengths corresponding to a reference spectrum,
wherein the glucose spectrum includes wavelengths having a higher absorption by glucose than the reference spectrum, and
wherein, in the glucose spectrum, an optical absorption in the human tissue varies in proportion to the glucose concentration in the human tissue;
a first optical sensor coupled to the housing, the first optical sensor configured to acquire optical intensity data in the first range of wavelengths of the glucose spectrum, the first optical sensor positioned to receive the light transmitted in a first direction from the human tissue when the housing is placed for use against the human tissue;
an optical filter for the first optical sensor, the optical filter positioned between the first optical sensor and the human tissue when the housing is placed for use on the human tissue, and the optical filter including a mixture of glucose and an optical potting material that is transparent in the first range of wavelengths;
a second optical sensor coupled to the housing, the second optical sensor configured to acquire optical intensity data in the second range of wavelengths of the reference spectrum, the second optical sensor positioned to receive the light transmitted in a second direction from the human tissue when the housing is placed for use against the human tissue;
a first optical barrier between the light source and the first optical sensor;
a second optical barrier between the light source and the second optical sensor; and
a processor configured to:
control illumination of the human tissue with the light source,
acquire a number of measurements including an intensity of the glucose spectrum at the first optical sensor and an intensity of the reference spectrum at the second optical sensor, and
calculate the glucose concentration in the human tissue based on a ratio of the number of measurements.

2. The system of claim 1, wherein the light source includes one or more light emitting diodes and a coating for the light source including a plurality of quantum dots configured to receive a first optical emission from the one or more light emitting diodes and emit a second optical emission in the glucose spectrum.

3. The system of claim 1, wherein the light source includes one or more light emitting diodes and a coating for the light source including a plurality of quantum dots configured to receive a first optical emission from the one or more light emitting diodes and emit a second optical emission in the reference spectrum.

4. The system of claim 1, wherein the first optical sensor and the second optical sensor include grayscale optical intensity sensors that acquire a single intensity measurement over a range of wavelengths including the first range of wavelengths and the second range of wavelengths.

5. The system of claim 4, wherein the processor synchronizes a first illumination in the glucose spectrum with a first intensity measurement with the first optical sensor, and a second illumination in the reference spectrum with a second intensity measurement with the second optical sensor.

6. The system of claim 1, wherein the optical potting material includes an optically clear silicone.

7. The system of claim 6, wherein the optical potting material includes a solvent for embedding glucose in the optical potting material.

8. The system of claim 1, wherein the second optical sensor is offset at a greater distance from a contact surface of the human tissue than the first optical sensor.

9. The system of claim 1, wherein the reference spectrum corresponds to an absorption spectrum of a first interfering molecule that absorbs light in the glucose spectrum as a function of concentration and has a variable concentration in the human tissue.

10. The system of claim 1, wherein the target spectrum includes a third range of wavelengths corresponding to at least one of a second glucose spectrum and a second reference spectrum.

11. The system of claim 1, wherein the housing includes a wearable physiological monitor.

12. A system for glucose detection, the system comprising:
a light source configured to emit light toward tissue of a subject, when the system is placed for use on a contact surface of the tissue of the subject, at a target spectrum including a first range of wavelengths corresponding to a glucose spectrum and a second range of wavelengths corresponding to a reference spectrum, wherein the glucose spectrum includes wavelengths having a higher absorption by glucose than the reference spectrum;
a first optical sensor positioned to receive the light emitted from the light source and transmitted by the tissue of the subject, the first optical sensor configured to acquire optical intensity data in the glucose spectrum;
a second optical sensor positioned to receive the light emitted from the light source and transmitted by the tissue of the subject, the second optical sensor configured to acquire optical intensity data in the reference spectrum;
an optical filter positioned between the first optical sensor and the tissue of the subject when placed for use, the optical filter configured to attenuate light transmitted to the first optical sensor within the glucose spectrum; and
a processor configured to calculate a glucose concentration in the tissue based on light intensity measurements from the first optical sensor and the second optical sensor.

13. The system of claim 12, wherein the light source includes a plurality of quantum dots tuned to emit light within at least one of the first range of wavelengths and the second range of wavelengths.

14. The system of claim 12, wherein the optical filter includes glucose embedded in an optically clear potting material.

15. The system of claim 12, wherein the processor calculates the glucose concentration based on a ratio of the light intensity measurements from the first optical sensor and the second optical sensor.

16. The system of claim 12, wherein the processor calculates the glucose concentration using at least one of a machine learning model, a calibrated linear regression model, and a lookup table.

17. A system for measuring concentration, the system comprising:
a light source configured to emit light toward a target material at a target spectrum when placed for use on a contact surface of the target material, the target spectrum including a first range of wavelengths corresponding to a measurement spectrum and a second range of wavelengths corresponding to a reference spectrum, wherein the measurement spectrum includes wavelengths having a higher absorption by a target molecule than the reference spectrum;
a first optical sensor configured to measure a first intensity of light transmitted by the target material in the measurement spectrum;
a second optical sensor configured to measure a second intensity of light transmitted by the target material in the reference spectrum;
an optical filter positioned between the first optical sensor and the target material when placed for use, the optical filter configured to attenuate the measurement spectrum more than the reference spectrum; and
a processor configured to calculate a concentration of the target molecule in the target material based on the first intensity and the second intensity.

18. The system of claim 17, wherein the target molecule includes water.

19. The system of claim 17, wherein the target molecule includes cholesterol.

20. The system of claim 17, further comprising a wearable physiological monitor that includes at least the light source, the first optical sensor, the second optical sensor, and the optical filter.
